(19) United States
(12) Patent Application Publication
(10) Pub. No.: US 2021/0154038 A1
(43) Pub. Date: May 27, 2021

(54) ORTHOSIS WITH A FABRIC LAYER AND A 3D PRINTED LAYER

(71) Applicant: Ford Global Technologies, LLC, Dearborn, MI (US)

(72) Inventors: Raphael Koch, Odenthal (DE); Lars Bognar, Dormagen (DE); Maik Broda, Würselen (DE)

(73) Assignee: Ford Global Technologies, LLC, Dearborn, MI (US)

(21) Appl. No.: 17/101,885

(22) Filed: Nov. 23, 2020

(30) Foreign Application Priority Data
Nov. 21, 2019 (DE) 102019131525.5

(51) Int. Cl. A61F 5/01; A41D 19/00; B29D 99/00; B33Y 10/00; B33Y 40/00; B33Y 80/00

(52) U.S. Cl. CPC A61F 5/0118; A41D 19/0006; B29D 99/0067; B29C 64/118; B33Y 40/00; B33Y 80/00; B33Y 10/00

(57) ABSTRACT

Orthoses are provided and methods for making orthoses. The orthosis is formed by providing a 3D printer, loading the 3D printer with a fabric layer, and loading a 3D printing material into the 3D printer. A 3D printing operation is performed wherein at least one 3D printed layer is 3D printed directly on the fabric layer. The fabric layer imprinted with the 3D printed layer is removed from the 3D printer.

### CROSS-REFERENCE TO RELATED APPLICATIONS

This application claims priority to and the benefit of German Application Number 102019131525.5, filed on Nov. 21, 2019. The disclosures of the above applications are incorporated herein by reference.

### FIELD

The present disclosure relates to a method for producing an orthosis, in particular an orthosis as a glove for use as an aid in manufacturing technology.

### BACKGROUND

The statements in this section merely provide background information related to the present disclosure and may not constitute prior art.

An orthosis is generally an aid that is used to stabilize, relieve, immobilize, guide or correct limbs or the trunk and that is produced industrially or by an orthopedic technician, often to a medical prescription.

Fused deposition modeling (FDM) or fused filament fabrication (FFF) generally designate a manufacturing method from the field of 3D printing, with which a workpiece is built up layer by layer from a fusible plastic or also from molten metal.

Orthoses for medical use or for use in manufacturing technology, in particular for use in serial production in the automotive industry, can be manufactured with the aid of 3D printing. Orthoses used in serial production allow the workers involved in said production to reduce the mechanical strain, for example, in loaded regions such as the finger joints. Orthoses are able to reduce a negative ergonomic effect, particularly during pressing-in operations or for lifting heavy objects. However, orthoses function in the desired manner only when they are perfectly adapted to the body of the user. Otherwise, injuries in the form of blisters or the like may arise. Most orthoses are therefore rather expensive, since they have to be manually adapted to each user. When orthoses are manufactured on a mass scale without individual customization, this has a negative effect as regards their suitable fit for the user. Orthoses produced by 3D printing can help create the perfect shape for each user individually. However, the rubber materials that are often used to achieve the flexibility of an orthosis are particularly susceptible to becoming brittle as time passes, which leads to failure of the orthosis.

In order to meet the various requirements, orthoses are typically manufactured from different materials. From the field of orthopedics, for example in Medical: plus medica OT—Maximum Flexibility and Design Freedom in the Production of Orthoses (retrieved on 22 Feb. 2019), methods for producing orthoses are known in which the plaster impression from the patient is digitized with a 3D scanner. The data are then transferred to a 3D printer which produces the orthosis layer by layer from a finely powdered material with the aid of a laser beam. The material used is based on a nylon polymer. In principle, provision is also made to design individual regions with different strengths and/or with different materials. Standard parts such as joint connections can likewise be integrated in the orthosis.

U.S. Pat. No. 9,610,731 B2 likewise relates to the production of an orthosis, in which a digital model of the orthosis is first established. With the aid of 3D printing, different regions of the orthosis can be configured with specific properties. Thus, individual regions of the orthosis can be stiff, and other regions can be flexible. Further components can be integrated in the orthosis during manufacture. In US 2016/0 374 431 A1, a comparable method is used for the purpose of configuring material properties such that, for example, a longitudinal expansion is specifically permitted in some regions.

U.S. Pat. No. 9,676,159 B2 discloses a printing device that can produce three-dimensional components from different materials. Individual materials can be applied directly onto the surfaces of textiles, for example, in particular of natural or synthetic fiber.

U.S. Pat. No. 10,016,941 B2 relates to a method in which a component for a shoe is manufactured with the aid of an FDM 3D printer, initially without the aid of support material. The component thus manufactured is then connected to further components of the shoe with the aid of a plug system.

An orthosis is also manufactured with the aid of an FDM 3D printer in US 2017/0 318 900 A1. For improved wearing comfort, the surface that forms the contact with the user has a substantially continuous area. The orthosis can be manufactured in this case from a single material, and, in addition to the manufacture by 3D printing, a fabric layer is placed onto a surface of the orthosis.

With the solutions disclosed in the prior art, it is not possible to make available an orthosis that can be produced cost-effectively in serial manufacture and that can nonetheless be individually adapted to the user during production.

### SUMMARY

The present disclosure provides a cost-effective orthosis and a method for serial production of such an orthosis that can nonetheless be individually adapted to the user during production.

It will be noted that the features and measures specified individually in the following description can be combined with one another in any desired technically meaningful way and disclose further refinements of the present disclosure. The description additionally characterizes and specifies the present disclosure, in particular in conjunction with the figures. When the user is mentioned in the description below, this includes both a worker who uses the orthosis, for example as an aid in assembly work in serial production in the automotive industry, and also a patient who uses the orthosis for medical purposes. “Inside” or “inner side” means that this side of the glove or of the orthosis is directed toward the user, while the “outside” or “outer side” is directed toward the environment of the orthosis.

The present disclosure relates to a method for producing an orthosis, in particular an orthosis as a glove for use as an aid in manufacturing technology, wherein the method has the following steps:

provision of a 3D printer;

provision of a fabric layer by loading the 3D printer with the fabric layer;

provision of a 3D printing material by loading the 3D printer with the 3D printing material;

performance of at least one 3D printing operation in order to form at least one 3D printed layer in the 3D printer directly on the fabric layer;

removal, from the 3D printer, of the fabric layer imprinted with the 3D printed layer.

The aforementioned steps, in one form of the present disclosure are performed in the sequence indicated. To produce an orthosis, individual method steps can also be performed several times. An orthosis with a fabric layer (e.g., composed of a textile fabric such as organza), and with a 3D printed layer specially adapted to the user is highly suitable in particular for use as a work glove in serial production or assembly work in the automotive industry. The fabric layer absorbs the user's perspiration, which may develop as a result of effort during physical work. Moreover, the fabric layer provides flexibility of an orthosis for work. At the same time, the 3D printed layer allows the orthosis to be tailored exactly to the intended field of use. FFF/FDM 3D printers are suitable as a 3D printer for a method according to the present disclosure, although the present disclosure is of course not limited to such 3D printers. For example, other methods are also suitable, e.g. pellet printing or granular printing to name but two, but these are not intended to be restrictive. The loading of a 3D printer with a fabric layer means that the fabric layer is inserted, drawn or placed into the 3D printer before a 3D printing operation or between two 3D printing operations. The 3D printing material for the 3D printed layer is made available, for example, by filament spools with correspondingly used starting material/3D printing material being inserted into the 3D printer. Depending on the starting material used, the 3D printing material thus generated has different mechanical properties and colors. By generating the 3D printed layer directly on the fabric layer, the production method can be simplified and the costs for producing an orthosis can be reduced. The generated 3D printed layer can be formed on the fabric layer in regions and/or can completely cover the fabric layer. In this way, reinforcements, clipping devices, shock absorbers, gripping aids or other specially designed regions can be made available on the fabric of a glove. Clipping devices or other coupling elements such as elastic bands, snap-fit closures, snap-fit connections or housing elements can likewise be provided in order to combine the 3D-printed hybrid orthosis with further fabric elements or other components.

Such a method for producing hybrid orthoses, i.e., orthoses with a fabric layer and a 3D printed layer, is not known from the prior art. An orthosis produced by a method according to the present disclosure can also be used in medical and/or therapeutic applications on patients.

In a variation of the method, the provision of the fabric layer by loading the 3D printer with the fabric layer takes place after a first 3D printing operation is carried out and before a second 3D printing operation is carried out in the 3D printer.

By virtue of the multi-layer design of the orthosis, the latter can be better adapted to the particular requirements. For example, during the production method, the 3D printer can be stopped after the first 3D printing operation. In a pause in the method, the fabric layer can then be inserted into the 3D printer onto a first 3D printed layer produced in the first 3D printing operation. Thereafter, in a second 3D printing operation, a second 3D printed layer is generated directly on the fabric layer. This operation can in principle be repeated as often as necessary.

After or before the removal, from the 3D printer, of the fabric layer imprinted with the 3D printed layer, the fabric layer may be cut out along a contour.

The cutting out of the fabric layer for the production or manufacture of the orthosis thus takes place either in the 3D printer or after removal from the 3D printer, for example by punching. The fabric layers can then be sewn to form a glove in such a way that the 3D printed layers are arranged exactly at the desired location, for example on a finger joint. Different sizes can also be produced in this way.

In an optional refinement of the method, it is possible, when performing the 3D printing operation and when forming the 3D printed layer in the 3D printer directly on the fabric layer, that an electronic component is formed in the 3D printed layer.

In this way, electronic components, namely active elements, such as switching devices, control knobs, sensors, or passive elements for receiving data, such as antennas or RFID elements, can be printed directly in one method step in the 3D printed layer. This permits the production of a digital glove. In order to realize more complex electronic components in the orthosis, the 3D printing operation can be stopped or paused such that electronic components, optionally implemented in a separate housing, can be placed in the 3D printer. A further 3D printing operation can then follow.

Independent protection is claimed for an orthosis, in particular an orthosis as a glove for use as an aid in manufacturing technology, in particular produced in one of the already described methods, having a fabric layer made of a fabric material and a 3D printed layer made of a 3D printing material, wherein the orthosis is produced by performing at least one 3D printing operation in the 3D printer in order to form at least one 3D printed layer directly on the fabric layer.

The 3D printed layer of the orthosis may be divisible into at least two regions, wherein a first region is flexible and/or bendable, for example by using flexible material such as thermoplastic polyurethane (TPU) or polyamide (PA), and a second region is stiffened and/or strengthened relative to the first region, for example by using fiber-reinforced material such as polylactides (PLA) or polyamide/carbon fiber (PA-CF).

By forming the 3D printed layer with different 3D-printable starting materials, different regions can be realized on the fabric layer. Flexible regions and stiffened regions, delimited from the flexible regions, can thus be realized on the fabric. Materials having an antibacterial action can also be printed onto the fabric layer.

In a variation of the orthosis, the 3D printed layer has a plurality of elongate printed layer elements, wherein spaces are or can be formed between the printed layer elements, such that the printed layer elements are movable relative to one another.

Through the formation of spaces between the individual printed layer elements, the degrees of freedom of the individual printed layer elements can be adjusted such that different translational movements or rotational movements of the printed layer elements relative to adjacent printed layer elements and to the fabric layer are permitted or not permitted. This provides that the movements of the user are carried out in an ergonomically precise manner, which inhibits undesired mechanical loading of the joints or tendons of the user. The printed layer elements, which are of elongate design, typically have a longitudinal axis, wherein the longitudinal axis is perpendicular to the fabric layer in the region of the connection element connecting the printed layer element to the fabric element. Two longitudinal axes of two adjacent printed layer elements can run parallel to each other, for example, or can enclose an angle corresponding to the curvature of the fabric layer. If a defined movement of the fabric layer is permitted, the orientation of the printed layer elements changes, and the angle that the longitudinal axes of the printed layer elements enclose with each other likewise changes.

The printed layer element of the 3D printed layer of the orthosis may have a longitudinal axis, a connection element assigned to the fabric layer, and a surface element assigned to the environment of the orthosis and/or to a further layer of the orthosis, wherein the printed layer element, starting from the connection element, tapers toward the surface element along the longitudinal axis.

In other words, the printed layer elements have the shape of a pyramid, a cone, a conical prism or a similar geometry which, starting from a base in the region of the connection element, tapers as far as its base in the region of the surface element. Tapers can also mean that the cross-sectional area perpendicular to the longitudinal axis of the printed layer element tapers from the connection element to the surface element. However, the tapering can also be configured the other way around, i.e., the cross-sectional area of the connection element is small compared to the cross-sectional area of the surface element.

In an optional refinement, the 3D printed layer of the orthosis has a metamaterial structure, wherein the metamaterial structure, under the effect of a tensile force in a first direction, expands in a second direction, wherein the first direction is different than the second direction and may be perpendicular to the latter. A metamaterial is generally a material that has unexpected properties compared to conventional material.

In addition, the orthosis, in particular the 3D printed layer of the orthosis, can have an integrated electronic component as a communications or computing device for transmitting, receiving, processing and/or storing data.

Electronic components can thus be integrated directly in the 3D printed layer. These include, for example, active elements such as switching devices, buttons or sensors, or passive elements such as printed antennas or RFID elements. By means of printed electronic components, a digital glove can be produced. For example, a glove can be produced with a scanning device, wherein the scanning device can scan a barcode for example, whereby the worker is able to identify the correct component. The glove can likewise comprise a switching device for operating the scanning device. In order to implement complex parts into the orthosis, the 3D printing operation can be interrupted or paused such that electronic components, optionally with an additional housing, can be placed into the 3D printer between two layers that are to be printed. It is also conceivable that, with communications or computing devices in the orthosis, possible collisions between the user and industrial robots can be indicated and inhibited. In addition to electronic components, further components can be integrated in the orthosis in order to couple the orthosis to further elements.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIGS. 1a and 1b each show a perspective view of examples of orthoses from the prior art;

FIG. 2 shows a schematic view of the layout of an orthosis according to the present disclosure;

FIGS. 3a-3b show a schematic view of an example of a design or arrangement of a 3D printed layer according to the present disclosure;

FIG. 4 shows a schematic view of a first alternative design or arrangement of a 3D printed layer according to the present disclosure;

FIGS. 5a-5b show a schematic view of a second alternative design or arrangement of a 3D printed layer according to the present disclosure;

FIG. 6 shows an example of an orthosis according to the present disclosure in a perspective view;

FIG. 7 shows an illustration of an example of a production method according to the present disclosure; and

FIG. 8 shows a flow chart of an example of a production method according to the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

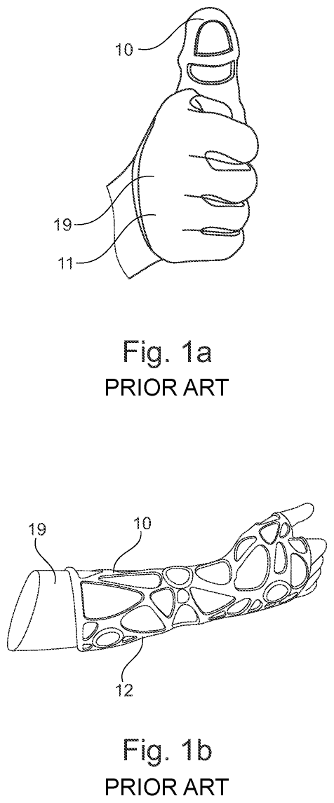

FIG. 1a shows an orthosis 10, designed as a thumb orthosis, from the prior art. The orthosis 10 is integrated in a glove 11. The thumb orthosis protects the thumb joints of the user 19, for example in assembly operations that are performed very frequently and that use a pressing-in movement of the thumb. This lessens the strain on the joints of the thumb. FIG. 1b shows an orthosis 10 from the prior art, designed as an exoskeleton 12 for the forearm and the palm. Such an orthosis 10 can also be used for medical purposes, and also as an aid that assists frequently performed movements, in particular in assembly operations, for example during serial manufacture on an assembly line in the automotive industry.

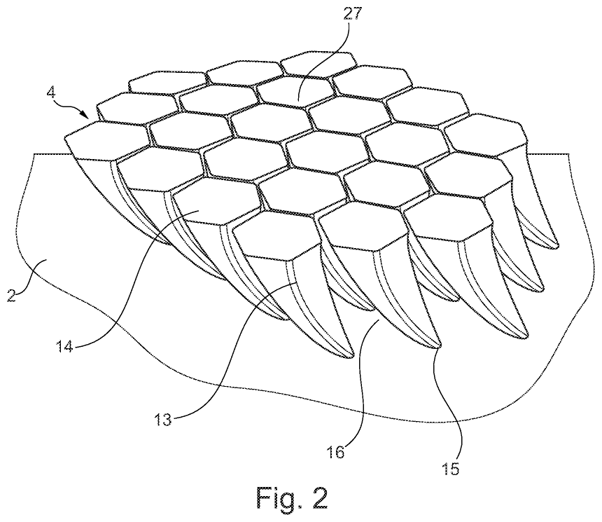

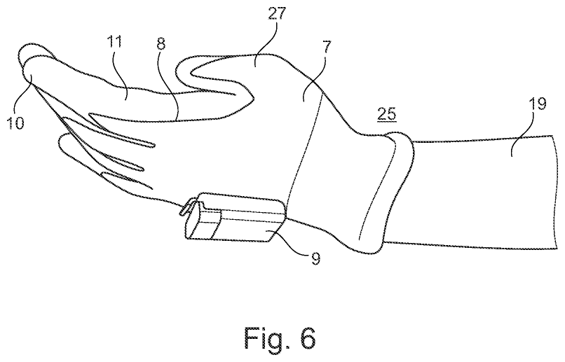

FIG. 2 shows a basic layout of the layers of an example of an orthosis 10 according to the present disclosure (cf. FIG. 6). A first layer or fabric layer 2 is provided which, during use of the orthosis 10, forms the contact with the skin of the user 19 (cf. FIG. 6) and thus also permits a comfortable feel when intensive effort causes sweating. A second layer or 3D printed layer 4 is applied onto the fabric layer 2. The 3D printed layer 4 has a multiplicity of printed layer elements 13, wherein the printed layer elements 13 are designed as a multiplicity of elongate components, i.e., pin-shaped or peg-shaped components. A printed layer element 13 has at least one surface element 14, for example for forming a contact with other objects, and a connection element 15 for connecting the printed layer element 13 to the fabric layer 2. Through the geometric formation of the printed layer elements 13, certain movements with the orthosis 10 by the user 19 (cf. FIG. 6) can be made more difficult or easier, depending on the requirements. By means of the surface elements 14 being arranged flush on one another, lateral movements of the printed layer elements 13 relative to one another can be made difficult. By means of a tapering, i.e., a reduction of a cross section starting from the surface element 14 as far as the connection element 15, spaces 16 form inside the 3D printed layer 4, which spaces 16 permit a desired freedom of movement of the user 19 (cf. FIG. 6). In other words, the fabric layer 2 may be directed toward the user (cf. FIG. 6), while the 3D printed layer 4, in particular the surface elements 14 thereof, is directed toward the environment 25 of the orthosis 10 and forms the outer side 27 of the orthosis 10. As can be seen from FIG. 2, by way of example, the surface elements 14 can be polygonal (e.g., hexagonal), and in a form is shaped like a honeycomb. The 3D printed layer 4 can be flexible and/or bendable, stiffened and/or strengthened, or a combination thereof (e.g., the 3D printed layer may be divisible into regions, and a first region may be flexible and/or bendable, and a second region may be stiffened and/or strengthened relative to the first region).

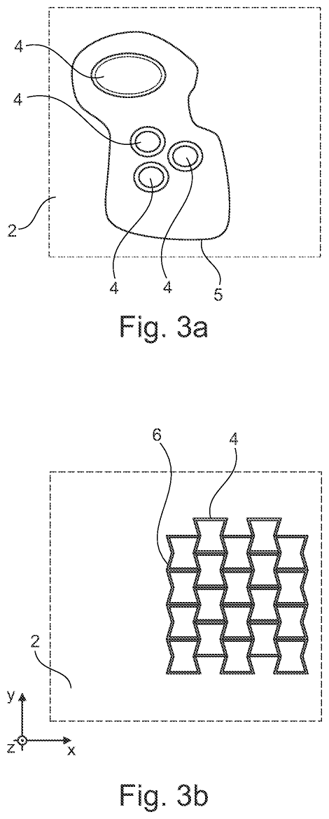

FIG. 3a shows a specific regional arrangement of a 3D printed layer 4 on the fabric layer 2. Alternatively, the 3D printed layer 4 can also be applied to the fabric layer 2 over the whole surface. To produce the orthosis 10 (cf. FIG. 6), a contour 5 can be applied to the fabric layer 2. The fabric layer 2 can be cut out along the contour 5. By subsequent cutting out of the fabric layer 2 after the application of the 3D printed layer 4 along the final contour 5, fraying of the fabric layer 2 that has taken place during the production method can be eliminated. FIG. 3b shows a further specific regional arrangement of a 3D printed layer 4 on a fabric layer 2, wherein the 3D printed layer 4 is formed at least partially with a metamaterial 6. Such a metamaterial 6, for example during an expansion of the 3D printed layer 4 in a y direction, can also bring about a lengthening of the fabric layer 2 in an x direction.

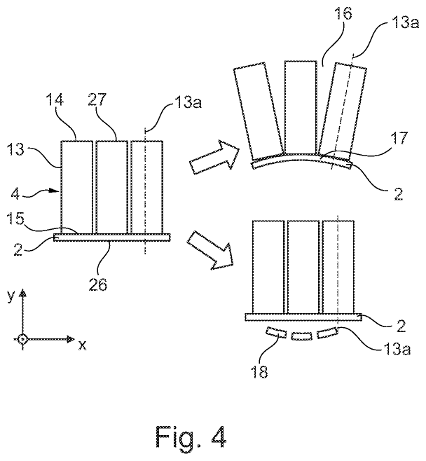

FIG. 4 shows a further possible variation of an arrangement of a 3D printed layer 4 on a fabric layer 2 and a possible design of the individual printed layer elements 13. Depending on the design of the individual printed layer elements 13, e.g., as pegs, small rods or pyramids, various degrees of freedom (DOF) of the fabric layer 2 and thus of the orthosis 10 can be realized during use of the orthosis 10. Starting from their surface element 14, the printed layer elements 13 are designed with an unchanging rectangular cross section as far as their connection element 15, for example with respect to the plotted y direction. By virtue of the unchanging cross section and the flush arrangement of the printed layer elements 13, no spaces 16 form between the individual printed layer elements 13 in their starting position, i.e., in their unloaded position. By means of this design there are admissible curvatures 17 of the fabric layer 2, i.e., the fabric layer 2 bulges outward away from the user 19 (cf. FIG. 6), that is to say in the direction of the environment 25 of the orthosis 10. The distance between the surface elements 14 of the individual printed layer elements 13 thus increases, and spaces 16 form between the printed layer elements 13. By contrast, a bulging movement in the opposite direction toward the inner side 26 constitutes an inadmissible curvature 18, wherein the inadmissible curvature 18 of the fabric layer 2 toward the inside, i.e., toward the user 19, is indicated by dashes as an imaginary line 13a. Since no spaces 16 are present between the printed layer elements 13 in their starting position, there is also no freedom of movement for an unwanted or inadmissible curvature 18.

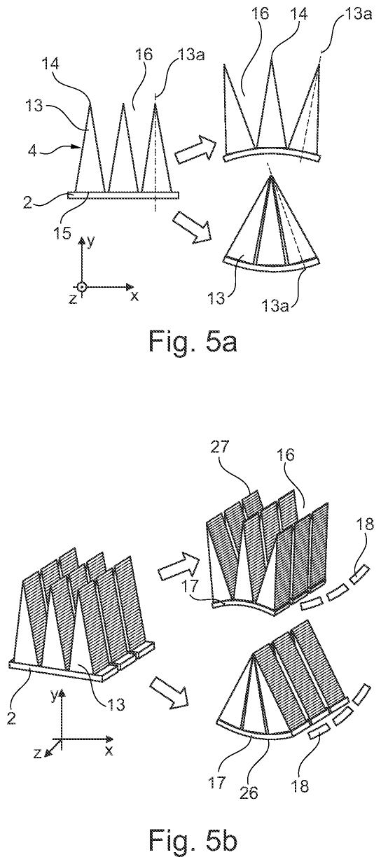

FIGS. 5a and 5b show a further possible variation of an arrangement of a 3D printed layer 4 on a fabric layer 2, in which, by means of a conical, pyramid-shaped and/or cone-shaped design of the individual printed layer elements 13, a bulging movement of the fabric layer 2 about the z direction is permitted in principle in both directions, i.e., to the inner side 26 and to the outer side 27. Starting from their connection element 15, the printed layer elements 13 taper as far as their surface element 14, for example with respect to the plotted positive y direction. Spaces 16 are thus formed between the printed layer elements 13. During an outward bulging movement of the fabric layer 2 about the z direction, i.e., in the direction of the environment 25 (cf. FIG. 6) of the orthosis 10, the spaces 16 enlarge as a result of the increase in the distance between the individual surface elements 14. During an inward bulging movement of the fabric layer 2 about the z direction, the distance between the individual surface elements 14 decreases, as a result of which the spaces 16 also grow smaller. This bulging movement can be continued until the printed layer elements 13 abut one another or bear flush on one another. The maximum bulging of the fabric layer 2 can be set by the geometric design of the printed layer elements 13. According to FIG. 5b, the printed layer elements 13 can also be designed such that the tapering is formed only with respect to the y direction, and not with respect to the z direction. The teachings illustrated by FIGS. 4 and 5a may be combined with each other. In this way, the individual printed layer elements 13 already lie flush or flat on one another with respect to the z direction, and an inadmissible curvature 18 with respect to the x direction caused by a bulging movement to the inner side 26 is thus suppressed. However, a curvature with respect to the x direction caused by a bulging movement to the outer side 27 in order to form spaces 16 is still possible. In this way, the individual DOF can be different depending on the direction.

FIG. 6 shows an orthosis 10, designed as a glove 11, on a user 19.

The orthosis 10 or the glove 11 has an inner side 26 (cf. FIG. 4) directed toward the user 19, and an outer side 27 directed toward the environment 25. To form the glove 11 as a digital glove 11, corresponding applications can already be incorporated in the production by 3D printing. For this purpose, the glove 11 can have various electronic components 7. For example, a scanning device 9, for example for scanning barcodes, can be provided in the glove 11, wherein the scanning device 9 can likewise be operated via a switching device 8 integrated in the glove 11.

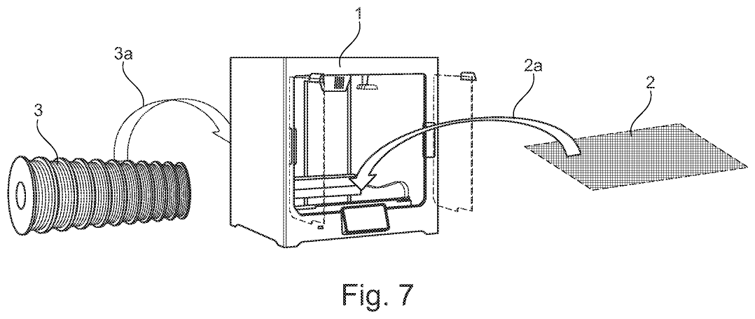

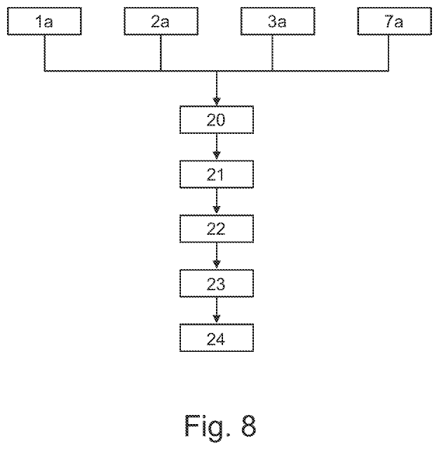

FIG. 7 illustrates an example of a production method according to the present disclosure for producing an orthosis 10 (cf. FIG. 6). A fabric layer 2 is provided 2a in an FFF/FDM 3D printer 1 by being secured in a platform of the 3D printer 1 such that slipping of the fabric layer 2 during the printing operation 20, 22 (cf. FIG. 8) is inhibited. A 3D printing material 3 is likewise provided 3a by inserting a filament spool 3 into the 3D printer 1.

FIG. 8 shows a method flow chart pertaining to an example of a production method according to the present disclosure for producing an orthosis 10 (cf. FIG. 6). Initially provided 1a, 2a, 3a, 7a are a 3D printer 1, a fabric layer 2, a 3D printing material 3 and, if appropriate, additional inserts, in particular non-3D-printable electronic inserts 7. After the fabric layer 2 has been inserted into the 3D printer 1, the 3D printing material is applied, in a first printing operation at 20, to the fabric layer 2 in order to form a 3D printed layer 4 (cf. FIG. 2). If appropriate, this first printing operation 20 can be interrupted by a pause at 21 such that, for example, electronic inserts 7, switching devices 8, scanning devices 9 and, if appropriate, further fabric layers 2 can be inserted. The 3D printing is then continued in a second 3D printing operation at 22. After completion of the 3D printing operation at steps 20 and 22, the orthosis 10 can be removed at 23, finished at 24, for example by cutting the fabric layer 2 to size along a contour 5 (cf. FIG. 3a), and then made ready by subsequent sewing.

Unless otherwise expressly indicated herein, all numerical values indicating mechanical/thermal properties, compositional percentages, dimensions and/or tolerances, or other characteristics are to be understood as modified by the word “about” or “approximately” in describing the scope of the present disclosure. This modification is desired for various reasons including industrial practice, material, manufacturing, and assembly tolerances, and testing capability.

As used herein, the phrase at least one of A, B, and C should be construed to mean a logical (A OR B OR C), using a non-exclusive logical OR, and should not be construed to mean “at least one of A, at least one of B, and at least one of C.”

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A method for producing an orthosis, the method comprising:
providing a 3D printer loaded with a 3D printing material;
loading a fabric layer into the 3D printer;
performing at least one 3D printing operation to form at least one 3D printed layer directly on the fabric layer;
removing, from the 3D printer, the fabric layer imprinted with the 3D printed layer; and
forming the fabric layer imprinted with the 3D printed layer into a glove for use as an aid in manufacturing technology.

2. The method according to claim 1, wherein the loading of the fabric layer into the 3D printer takes place after a first 3D printing operation is performed and before a second 3D printing operation is performed in the 3D printer.

3. The method according to claim 1, wherein, after and/or before removing, from the 3D printer, the fabric layer imprinted with the 3D printed layer, cutting the fabric layer along a contour.

4. The method according to claim 1, wherein when performing the 3D printing operation and when forming the 3D printed layer in the 3D printer directly on the fabric layer, an electronic component is formed in the 3D printed layer.

5. The method according to claim 1, wherein when performing the 3D printing operation and when forming the 3D printed layer in the 3D printer directly on the fabric layer, forming a metamaterial in the 3D printed layer.

6. The method according to claim 1, wherein when performing the 3D printing operation and when forming the 3D printed layer in the 3D printer directly on the fabric layer, 3D printing a plurality of individual printed layer elements.

7. The method according to claim 6, wherein the plurality of individual printed layer elements inhibit the degrees of freedom of movement of a user.

8. A method for producing an orthosis, the method comprising:
providing a 3D printer loaded with a 3D printing material;
loading a fabric layer into the 3D printer;
performing at least one 3D printing operation to form at least one 3D printed layer directly on the fabric layer; and
removing, from the 3D printer, the fabric layer imprinted with the 3D printed layer, wherein the at least one 3D printed layer comprises an electronic component.

9. The method according to claim 8, wherein the loading of the fabric layer into the 3D printer takes place after a first 3D printing operation is performed and before a second 3D printing operation is performed in the 3D printer.

10. The method according to claim 8, wherein, after and/or before removing, from the 3D printer, the fabric layer imprinted with the 3D printed layer, cutting the fabric layer along a contour.

11. The method according to claim 8, wherein when performing the 3D printing operation and when forming the 3D printed layer in the 3D printer directly on the fabric layer, forming a metamaterial in the 3D printed layer.

12. The method according to claim 8, wherein when performing the 3D printing operation and when forming the 3D printed layer in the 3D printer directly on the fabric layer, 3D printing a plurality of individual printed layer elements.

13. The method according to claim 12, wherein the plurality of individual printed layer elements inhibit the degrees of freedom of movement of a user.

14. A glove orthosis comprising:
a fabric layer; and
a 3D printed layer formed over at least a portion of the fabric layer, wherein the 3D printed layer comprises an electronic component.

15. The glove orthosis according to claim 14, wherein the 3D printed layer is divisible into at least two regions, wherein a first region is flexible and/or bendable, and a second region is stiffened and/or strengthened relative to the first region.

16. The orthosis glove according to claim 14, wherein the 3D printed layer has a plurality of printed layer elements, wherein spaces are formed between the printed layer elements, such that the printed layer elements are movable relative to one another.

17. The orthosis glove according to claim 16, wherein the printed layer element of the 3D printed layer of the orthosis has a longitudinal axis, a connection element connecting the printed layer element to the fabric layer, and a surface element exposed to an environment of the orthosis, wherein the printed layer element extends from the connection element to the surface element along the longitudinal axis.

18. The glove orthosis according to claim 16, wherein the printed layer elements are elongated.

19. The glove orthosis according to claim 14, wherein the 3D printed layer comprises a metamaterial structure, wherein the metamaterial structure, under the effect of a tensile force in a first direction (y direction), expands in a second direction (x direction).

20. The glove orthosis according to claim 14, wherein the electronic component is an integrated electronic component comprising a communications or computing device for transmitting, receiving, processing and/or storing data.
